(19) United States
(12) Patent Application Publication
(10) Pub. No.: US 2008/0221469 A1
(43) Pub. Date: Sep. 11, 2008

(54) FITTING AND FLUID-CONVEYING DEVICE CONNECTED THERETO

(76) Inventor: George John Shevchuk, Lakewood, NJ (US)

Correspondence Address: DINSMORE & SHOHL LLP, ONE DAYTON CENTRE, ONE SOUTH MAIN STREET, SUITE 1300, DAYTON, OH, 45402-2023, US

(21) Appl. No.: 12/041,946

(22) Filed: Mar. 4, 2008

Related U.S. Application Data
(60) Provisional application No. 60/893,728, filed on Mar. 8, 2007.

(51) Int. Cl. A61B 5/097; B01D 63/06; F16L 33/18

(52) U.S. Cl. 600/532; 96/10; 285/242; 285/243

(57) ABSTRACT

A fitting for a fluid-conveying conduit. The fitting includes integrally-formed clamps that can be used to secure the conduit in a locking relationship so that likelihood of leakage and separation of the fitting and the conduit is reduced. In one form, the fitting and conduit can be used as a moisture exchange device. In a more particular form, the conduit wall of the moisture exchange device includes a water-permeable material such that a humidity level in the conduit can be controlled. Since the fitting is formed separately from the end of the conduit to which it is to be attached, fabrication cost and complexity is reduced.

This application claims the benefit of U.S. Provisional Application Ser. No. 60/893,728, filed Mar. 8, 2007.

### BACKGROUND OF THE INVENTION

The present invention relates generally to a fitting for a fluid conduit, and specifically to a fitting that is formed separately from the conduit and readily attachable thereto.

Flexible fluid-conveying conduit, such as tubes, hoses or the like, is used in various applications to promote the secure, efficient and leak-free transport and handling of a gas, liquid or combination of both between a fluid source and destination. Examples include tubing used for chemical and related industrial processes, as well as for medical equipment and procedures, many of which require tight controls on the amount of humidity and related moisture present in a fluid flowing through a conduit. Other examples include hoses for water transport, such as garden hoses, potable water supplies or the like. In addition to the aforementioned applications, such conduit may be used for fire and gas detection, environmental control, safety and security equipment. A typical fluid-conveying tube, hose or associated conduit may employ an extrudable or related material, such as metal or plastic.

One area where plastic fluid-conveying tubes are used to reduce the humidity level of a fluid flowing through it is in the medical device field, examples of which include ventilators, medication dispensers (such as anesthesia equipment) and analysis equipment. A particular application involves a breathing tube dryer system, where one end of the tube may be connected to a patient's nose, mouth or other breathing orifice, with the other connected to a gas analyzing machine. Such analysis necessitates clean, dry gas samples in order to ensure accurate readings of constituent gases. In prior U.S. Pat. Nos. 3,735,558 and 4,705,543, owned by the assignee of the present invention and herein incorporated by reference, lower humidity levels could be achieved by constructing the tube wall out of a water-permeable material such that excess water present in the fluid flowing through the tube can diffuse through the tube wall to the ambient air with a lower water concentration. The selective nature of a properly-constructed permeable material is such that only water is removed from the fluid flowing through the tube, leaving other constituents in the gas stream. This is especially valuable in situations where the fluid is being analyzed for constituent make-up, where the presence of such water can lead to decreased sensitivity and increased maintenance of analyzers and related gas-sampling equipment. In addition to the aforementioned drying, there may be circumstances where additional humidity is necessary, and the nature of the permeable tubing wall is such that either can be readily achieved.

One material particularly well-suited to drying or humidifying a gas stream is the class of materials known as perfluorinated ionomers, particularly Nafion®, a perfluorosulfonate ionomer that, due to the presence of sulfonic acid groups in the polymer, is extremely effective in permeating water through ionic channels from one surface to the other. In U.S. Pat. No. 4,705,543, as well as U.S. Pat. No. 6,779,522 (also owned by the assignee of the present invention and hereby incorporated by reference), Nafion® or related water-permeable tubing is made very thin (for example, between 0.002 and 0.003 inches thick) to promote a perevaporation process. An outer layer of a mesh material (for example, in a woven, braided or formed configuration) may also be used to enhance the tube's mechanical integrity. Unlike diffusion, which occurs rather slowly due to the permeation of a microporous membrane, the process using Nafion® occurs as a water-of-hydration approach, and because of this first order kinetic reaction, the desired humidity level (whether higher or lower) can be achieved rapidly (for example, within two tenths of a second or less). Moreover, the water absorption and subsequent pervaporization proceeds with no net change in free energy. As such, no additional energy (other than the aforementioned concentration gradient) is needed to drive the reaction, thereby enhancing tube simplicity.

In a typical fluid-conveying tube that employs an extrudable or related plastic material, the ends of the tube can be terminated with fittings (also known as connectors), thereby allowing a preferred length of tube to be formed. The fitting facilitates connection between the tube and a tube-receiving receptacle to promote a secure fit between them. In one configuration, the fitting can be attached to the tube ends, examples of which include internal metal (such as stainless steel) couplings, compression fittings, overmolded headers or the like. In configurations involving fluid drying tubes (such as the aforementioned breathing tubes), the fittings promote ease of connection to a patient's breathing mask on one end and gas analyzer at the other end. Preferably, the fittings form a gas-tight seal for fluid containment integrity, as well as locking features for mechanical integrity of the connection. Heat shrinking or related processes can be used to connect these fittings to the tube.

The overmolded header configuration with a metallic inner coupling has been particularly used because of its durable construction. Referring initially to FIG. 2 in conjunction with FIG. 1, a fitting 100 according to an aspect of the prior art is overmolded onto a moisture exchange tube 10 (also referred to as a fluid drying tube), which is made up of a water-permeable tube 20 (preferably, although not necessarily, made of Nafion® or related material), braiding 30 to provide support to the water-permeable tube 20, and an optional coupling tube 40 configured to fit inside the water-permeable tube 20 to provide inner support of the water-permeable tube 20 and the overmolded fitting 100. The overmolded fitting 100 includes an elongate cylindrical body 110 formed concentrically around the braiding 30, as well as an integral barb 120 at the end. The male barb 120 includes a radial projection to promote secure fit between the fitting 100 and a complementary socket or second plastic tube (not shown). A fluid bore 130 extends through the body 110 to define a flowpath that connects to water-permeable tube 20.

While the overmolded design of FIG. 2 has many benefits, its construction entails numerous steps, which increase complexity, cost and the likelihood of rejected finished parts. Furthermore, while barbed fittings are common in the industry for connecting tubing for low-pressure applications, where simply inserting a barbed fitting into a tube is sufficient to ensure leak-free connection, their use is limited in situations where higher pressures or resistance to being pulled apart is present. In such applications, separate clamps are typically employed to provide additional fitting reinforcement. Unfortunately, separate clamps require additional part tracking and inventorying, and necessitate the use of tools (including pliers, pincers, crimpers, screwdrivers or the like) to connect them to the tube. Such adds cost and complexity to the clamping process.

Accordingly, it is desirable that a fitting be provided that can be formed separately, yet provide self-clamping to achieve a secure, connection to a fluid handling tube, hose or similar conduit to which it is coupled to decrease the likelihood if inadvertent separation of such tube, hose or similar conduit from another tube to which the fitting is attached. It is additionally desirable that the fitting have additional securing features built into it to allow for increased mechanical properties. It is further desirable that such a fitting be inexpensive and easy to manufacture and couple to the tube, hose or similar conduit.

### BRIEF SUMMARY OF THE INVENTION

According to a first aspect of the invention, a fitting for securing to the end of a tube, conduit or other fluid handling container is disclosed. The fitting is made up of a generally elongate body that defines a central fluid bore through its axial dimension. The body includes a fluid inlet and first connector defining a proximal end of the fluid bore, a fluid outlet with a second connector defining a distal end of the fluid bore such that the inlet and outlet are in fluid communication with one another. The body additionally includes numerous clamps that are integrally formed with the body through a hinged connection. In this way, the integral formation of the clamps to the fitting precludes the need for the separate reinforcing clamps and concomitant tooling discussed above. By the present construction, when the clamps engage with one another, they force the tube to be compressed to frictionally engage with a respective one of the first and second connectors on the fitting body. This increases the resistance to inadvertent separation between the fluid handling tube and the connectors. By the present construction, the bore forms a portion of a flowpath defined in the tube, which is beneficial in reducing the likelihood of leakage between fluidly coupled tubes that may otherwise have a discontinuity in the flowpath between them. The construction of the fitting is additionally beneficial over fittings that merely act as external connectors without any internal tube support in that by having connector ends that couple to the inner surface of the tube, they provide (in addition to a continuity of flowpath with the tubes and other devices that are fluidly engaged with the first and second connectors) internal support against the compressive forces imparted to the tube by the clamps. As can be seen, the fitting is of a one-piece (i.e., unitary) structure that can be formed from molded plastic or related resilient material.

Optionally, the plurality of clamps comprise a pair of clamps. In a more particular option, the pair of clamps form a resiliently-biased locking relationship with one another through at least one tab formed on one of the pair of clamps that engages with a complementary slot formed in the other of the pair of clamps. Even more particularly, at least one of the pair of clamps comprises one or more tooth-like projections disposed on a fluid handling tube-engaging surface thereof, the projection configured to impart a gripping contact with the outer surface of the fluid handling tube. In another option, the pair of clamps extend from the body such that they are substantially radially-opposed to one another. More particularly, the projections formed on the inner surface of the pair of clamps are cooperative to form a concentric connection around the body. In another option, the projections extending from the pair of clamps are cooperative to form a generally rectangular connection around the body. In this latter form, rather than fostering a frictional contact over a relatively large surface area of the tube, the tooth-like projections dig into or create indentations in relatively discreet parts of the surface of the tube. In yet another option, at least one of the first and second connectors comprises a barbed end to increase connectivity between the fitting and the tube to which it is attached. In a particular form, the barbed end (or ends) defines a frustro-conical cross-sectional shape. Variations on barbed ends are also available so that luer fittings or similar constructions are possible. One particular application of the presently-disclosed fitting is for use in a fluid drying tube, such as a breathing tube or the like where the moisture content of the fluid needs to be reduced.

The fittings described herein can be used in myriad applications that involve tubing, hosing or related fluid conduits, with sizes dictated according to the dimensions of the conduit. Thus, in addition to moisture exchange tubing (such as breathing dryer and humidifier tubes), such fittings can be used for other industrial and fluid-processing equipment, such as sample conditioning systems, thermoelectric coolers, filters, scrubbers and probes connected to the same. Simple water-conveying hoses and associated equipment can also benefit from an appropriately-sized fitting according to the various aspects of the present invention. Likewise, uses for fire and gas detection, environmental control, safety and security equipment and various fluid conveyance applications are also within the scope of the device of the present invention. By extending the length of the clamps along their axial dimension, the fitting can be made to function as a coupler by having both body ends able to be engaged by the clamps, thereby promoting both fluid connectivity and additional resistance to inadvertent tube separation.

According to another aspect of the invention, a fluid-conveying apparatus is disclosed. The apparatus includes a fluid tube and a fitting coupled to the tube. The fitting includes a generally elongate body defining a fluid bore therethrough such that the bore forms a portion of a flowpath defined in the tube. The body includes a fluid inlet at a proximal end of the fluid bore and a fluid outlet at a distal end of the fluid bore. The inlet and outlet are in fluid communication with one another such that together they can make up part of the flowpath when connected to the tube. As with the previous aspect, numerous clamps that are integrally formed with the body through a hinged connection are also included so that when the clamps are brought into their closed (i.e., clamped) position, they compress the tube onto a respective one of the proximal and distal ends of the fitting body. As previously stated, such tight, compressed fit promotes a connection between the tube and the fitting such that resistance to inadvertent separation is reduced, as well as with another fluid-conveying device (for example, another tube) that is coupled to the other of the proximal and distal ends.

Optionally, the tube is configured as a moisture exchange tube comprising a selective and reversible water-absorption inner member, and an outer member disposed about the inner member. More particularly, the outer member may be configured to permit direct fluid contact between the inner member and the air or other ambient environment. Even more particularly, the outer member comprises a braided netting to facilitate both external support of the inner member as well as moisture exchange between the ambient environment and the fluid being conveyed through the inner member. In another option, the tube is a component in a medical device, such as a breathing tube, medical dryer or other component where the fluid being conveyed through the tube has its humidity or related property adjusted by the interaction between the tube and the ambient environment. The tube may also be a component in non-medical applications as well, including gas sample conditioning or processing equipment.

As previously discussed, the water-permeable wall of the tube inner member may be made from Nafion® or a related material that promotes a first order kinetic diffusion reaction. Also as previously discussed, resilient snap-fit features of the fitting clamps allow the fitting to be quickly coupled to a tube. The choice of materials, such as plastic, means the tube and fitting can be a disposable device. This may be particularly beneficial when the apparatus is part of a breathing tube or other medical device, where concerns about sanitary conditions may be paramount. One such use may be in capnography to monitor the concentration or partial pressure of carbon dioxide in respiratory gases, especially during intensive care procedures or such times where anesthesia may be used. During anesthesia, a capnogram provides direct indicia of the elimination of carbon dioxide by the lungs to the anesthesia device, and the device of the present invention can be used to improve such a procedure.

According to another aspect of the invention, a method of assembling a fluid-conveying device that includes the fitting and a tube or related fluid-conveying conduit is disclosed. In a related aspect, a method of using a fluid-conveying device is disclosed. In another related aspect, a method of placing a terminating fitting at the end of a fluid-conveying tube is disclosed. In another aspect of the invention, a method of attaching a fluid-conveying conduit is disclosed. The method includes configuring a fitting to comprise a generally elongate body defining a fluid bore therethrough such that the bore forms a flowpath, the body comprising a fluid inlet defining a proximal end of the fluid bore and a fluid outlet defining a distal end of the fluid bore and in fluid communication with the fluid inlet, and a plurality of clamps integrally formed with the body through a hinged connection; and connecting the fitting to the conduit such that a flowpath formed in the conduit is placed in fluid communication with the fitting flowpath, further such that upon engagement of the plurality of clamps with one another, the plurality of clamps cause the conduit to compressively engage with a respective one of the proximal and distal ends of the fitting such that the conduit is better secured.

In one option, the conduit is a tube, such as a breathing tube, dryer tube or the like. Specifically, the tube may be a moisture exchange tube with a water-permeable member formed therein. The clamps may be configured as discussed in the previous aspects, including having the clamps that are substantially radially-opposed to one another form a resiliently-biased locking relationship with one another to make a concentric connection around the body of the fitting. Likewise, the projections can be formed such that they define (in a first configuration) a generally circular-shaped inner surface by the joined clamps and (in a second configuration) a generally rectangular-shaped inner surface. One way such rectangular-shaped inner surface is enabled is by having the projections on each clamp define a generally V-shaped tube-engaging surface. The construction of the fitting is such that the method of attaching may be accomplished without the use of tools.

### BRIEF DESCRIPTION OF THE SEVERAL VIEWS OF THE DRAWINGS

The following detailed description of the present invention can be best understood when read in conjunction with the following drawings, where like structure is indicated with like reference numerals and in which:

FIG. 1 shows the end of a moisture exchange tube prior to the application of a fitting according to an embodiment of the prior;

FIG. 2 shows a fitting that has been overmolded onto the moisture exchange tube of FIG. 1;

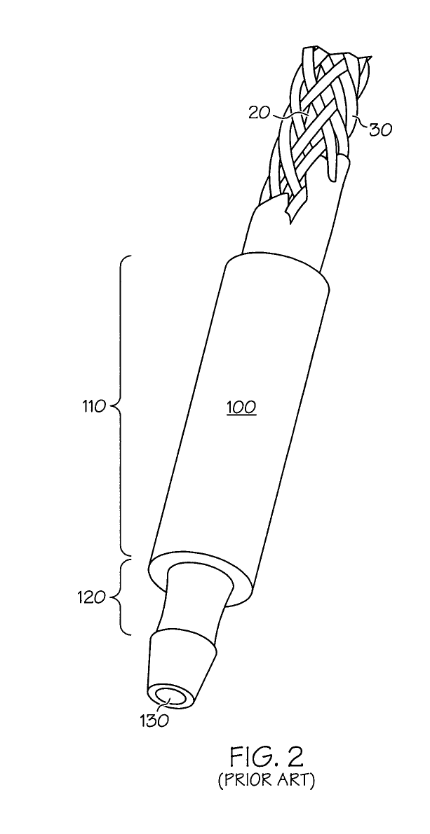

FIG. 3 shows a view of the fitting according to an embodiment of the present invention;

FIG. 4A shows the fitting of FIG. 3 in its unsecured position prior to attachment to a moisture exchange tube;

FIG. 4B shows the fitting of FIG. 4A joined to the end of, but not yet secured to, the moisture exchange tube;

FIG. 4C shows the clamps of the fitting of FIG. 4B being brought into cooperation with one another to secure the fitting to the moisture exchange tube; and

FIG. 5 shows a modified embodiment of the fitting of FIG. 3 with tubes attached at both proximal and distal ends.

### DETAILED DESCRIPTION OF THE PREFERRED EMBODIMENTS

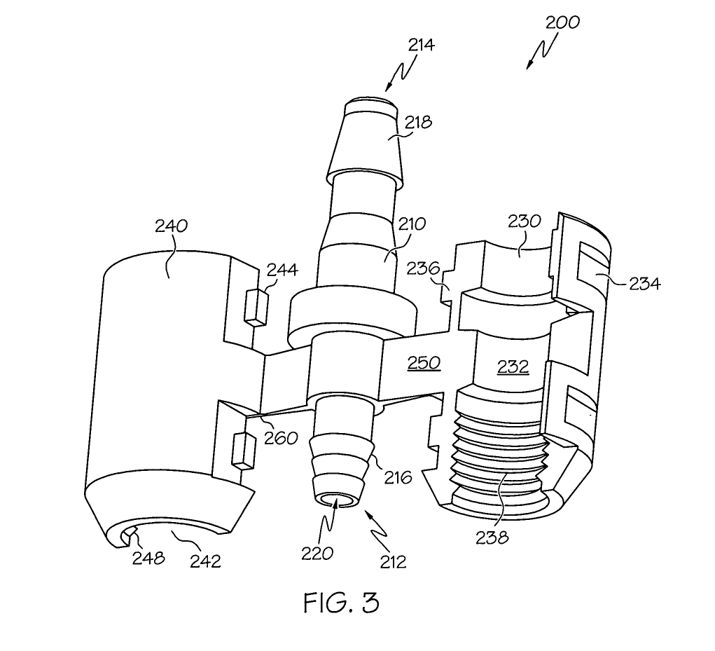

Referring to FIG. 3, a fitting 200 according to an aspect of the present invention has a body 210 comprising a proximal end 212 and a distal end 214 between which a fluid bore 220 extends to establish fluid communication between a first tube (not presently shown) and a socket, second tube or related receptacle (none of which are shown, and collectively referred to as a second tube, a component, a device or the like, the usage of which will be apparent from the context). A fluid inlet corresponds to the proximal end 212, while a fluid outlet corresponds to the distal end 214, although it will be appreciated by those skilled in the art that the terms “inlet” and “outlet” are relative, and that in the event of a reversal of flow through a first and second fluidly-coupled tubes, the roles would be reversed. Each of the proximal and distal ends 212, 214 include radially-projecting male barbs 216, 218 that act as connectors and can be used to engage complementary surfaces on the respective mating first and second tubes. As shown, the barbs 216, 218 are of a general frustro-conical construction so that in a preferred form, the second tube would slide over the barb 218 to engage body 210 in a friction-fit relationship, while the first tube would slide over the barb 216 to engage body 210 in a similar friction-fit relationship.

Fitting 200 additionally includes a pair of radially-opposed semicircular clamps 230,240. The clamps 230, 240 are laterally and integrally-formed to body 210 through radially-extending bridges 250 that project from a substantial centerline of the body 210 to a substantial centerline of each of the pair of clamps 230, 240. A hinge is formed by a frangible line of weakness 260 in the region between bridge 250 and each of the respective clamps 230, 240 to allow them to be folded in a radially-inward direction onto the outer surface of an engaging tube or related receptacle (neither of which are shown) that is frictionally-fit onto the body 210 over proximal end barbs 216 as described above. The inner surfaces 232, 242 form the tube-engaging surfaces in that at least a portion of the inner surfaces 232, 242 will compressively contact the outer surface of the tube. To better engage the tube, the inner surfaces 232, 242 are shaped with tube-engaging projections 238, 248 near the proximal end such that when brought into facing relationship with one another, they together generally force the engaging tube (which is preferably made from a deformable material such as plastic, rubber or the like) to conform to the shape defined by the inner surfaces 232, 242. As shown, each of the individual projections 238, 248 define a generally sawtooth-shaped profile to improve the ability to “bite” or otherwise engage tube 10, although it will be appreciated that other profiles, such as squared or rounded, may also be employed. In addition, the projections 238, 248, when placed adjacent one another upon engagement of clamps 230, 240, produce generally circular-shaped inner surface in the fitting 200. Alternate gripping shapes could be anticipated in the filing, for example, using spiked shapes instead of one with projections. Another exception deals with the profile defined by the projections 338, 348. Slots 234 formed on the radially-outward part of clamp 230 are sized and shaped to accept complementary detents or tabs 244 formed on the radially-inward part of clamp 240, while detents or tabs 236 formed on the radially-inward part of clamp 230 can form a similar locking relationship with slots (not shown) formed on the radially-outward part of clamp 240. This latched cooperation between tabs and slots facilitates a locking relationship between the clamps 230, 240. Such locking relationship can be resiliently biased in circumstances where at least one of the tabs or slots are formed from a deformable material, such as a plastic. Regardless of the nature of the locking relationship, the clamping connection of the fitting 200 to the tube or related receptacle provides a way to couple adjacent first and second fluid-conveying tubes, devices or related containers in such a way to decrease the likelihood of inadvertent separation. In the present context, the other fluid-conveying device may include fluid processing machinery, such as those used for anesthesia and related medical procedures, as well as similarly-functioning industrial or process machinery.

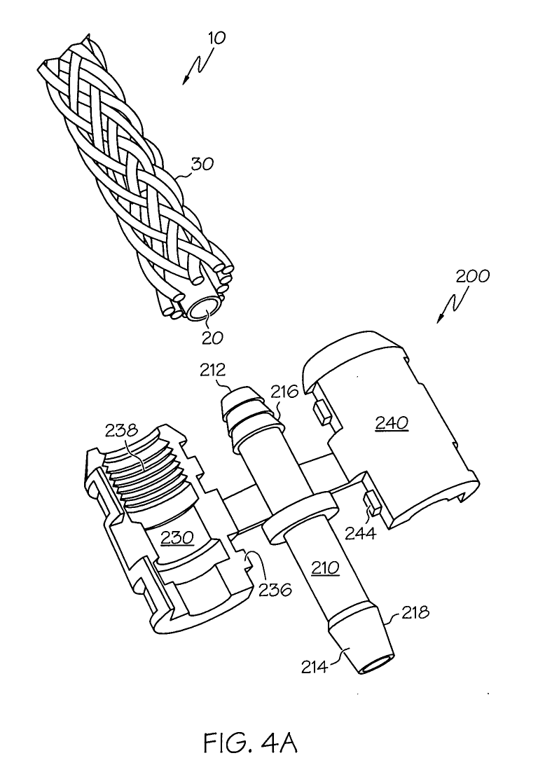

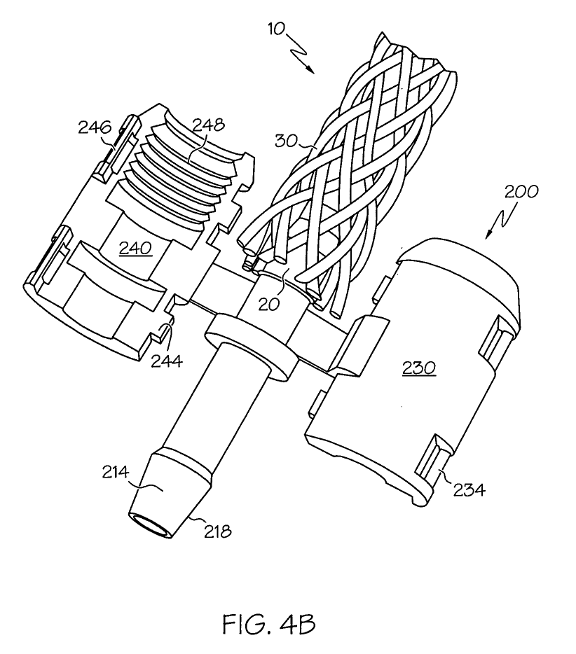

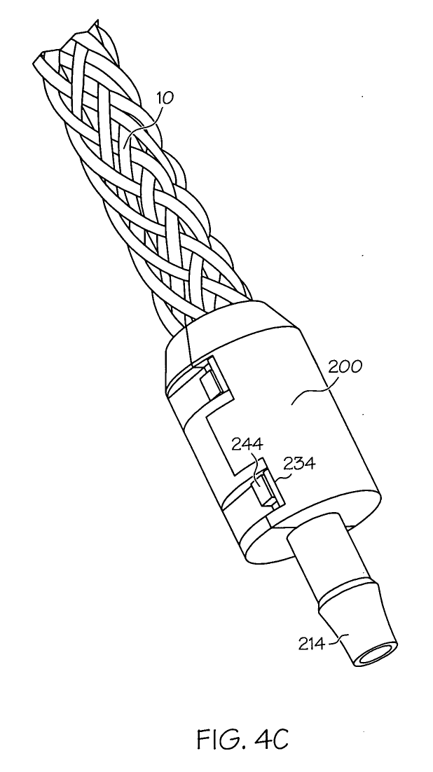

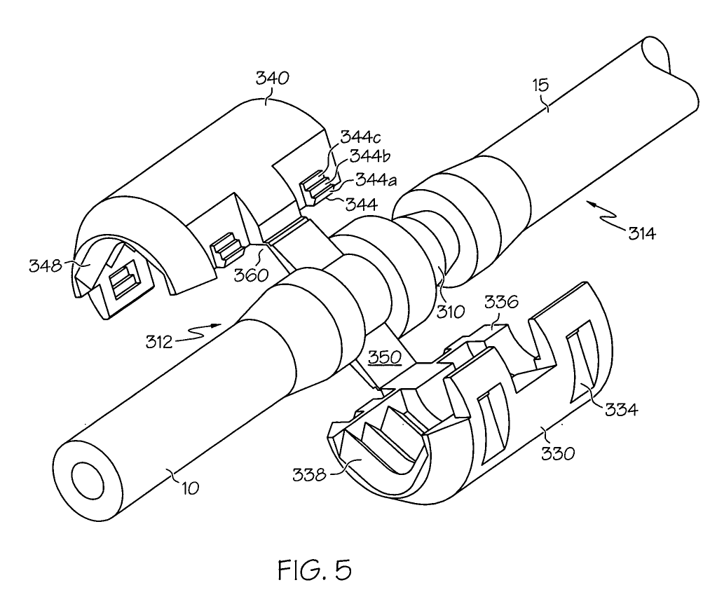

Referring next to FIG. 5, a fitting 300 that is a variation of the fitting 200 of FIG. 3 is shown. In most respects, its features are similar to those of fitting 200. One exception deals with the latched cooperation between the clamps 330 and 340. As presently shown, detents 344 include a stepped (or ridged) construction 344A, 344B and 344C such that, depending on the diameter of the tube 10 placed over a corresponding proximal end 312 of the fitting 300, passes all or part of such steps into complementary slot 336. Stepped detents 344A, 344B and 344C allow for a ratcheted connection between the opposing clamps 330, 340. It will be appreciated by those skilled in the art that other shape variations are possible, and as such within the scope of the present invention. Unlike the embodiment depicted in FIGS. 3, 4A and 4B, where the projections 238, 248 associated with each respective clamp half 230, 240 form a semicircular profile, the profile formed by projections 338, 348 is V-shaped (best shown in clamp 330 in FIG. 5) to permit selective engagement of the projections 338, 348 with the outer surface of tube 10. The V-shaped nature of their profile means that when the projections 338, 348 are placed adjacent one another through the engagement of the opposing clamps 330, 340, the projections 338, 348 produce a generally rectangular-shaped inner surface in the fitting 300, where a particular form of the inner surface is square-shaped. Such V-shaped feature of the projections 338, 348 is particularly well-suited to the ratcheting detent variation depicted in FIG. 5, as such improves the ability to attach the fitting 300 to a variety of different tube 10 diameters rather than being designed for a specific one. The apex of the projection 338, 348 always engages the tubing in at least four localized areas (two per each clamp 330, 340).

The present inventor has conducted tests on three versions of barb prototypes that were tooled and molded using Capron® 8202 nylon. When using some braided covering materials (such as polypropylene), the strength of the external engagement may be able to be augmented, such as through the use of ultrasonic welding or the like. In fact, this may be either in addition to the latched cooperation between detents and slots or a replacement for them. Also, alternate gripping shapes used to make stronger latches and firmer grip of outer latches onto the dyer braiding could be employed. Using the assembled barbs on each end, the inventors were able to support over 3.5 lbs. of applied force before movement of the braiding out of the barb clamp was observed. Additional samples were assembled, and then ultrasonically welded across the fittings' joints. A pull force of 8 lbs. was reached before the barb clamped in the gripping fixture was stripped. No movement of the braiding relative to the fitting body was observed, thereby indicating that the welded fittings should be able to handle any reasonably-expected pull force requirement, while the un-welded ones may well be adequate for most customer applications. An additional pull test was conducted using Tygon® tubing with a 1/16″ inner diameter and ⅛″ outer diameter, where, for comparison purposes, a standard industry barb designed for this size tubing provided a pull strength of 4.5 lbs before the fitting pulled out. The smaller diameter barb of the new fitting provided only about ½ lb of retention strength to this tubing by itself; however, with the external clamps engaged, the new fitting provided 6.5 lbs of retention force. Thus, the present design is useful with standard soft tubing where additional retention force over that provided by simple barbs is desired.

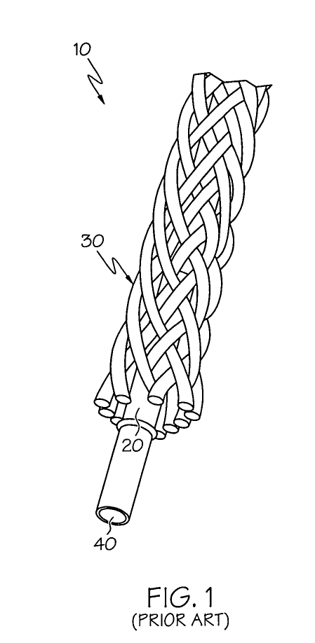

Referring next to FIGS. 4A through 4C, a first tube (shown as moisture exchange tube 10) and a fitting 200 attachable to the first tube are shown. The moisture exchange tube 10 presently shown is generally similar in construction to the moisture exchange tube 10 of FIG. 1 except for the removal of the support tube 40, which is no longer needed. As shown with particularity in FIGS. 4A and 4B, the end of the water-permeable tube 20 (also referred to as the water permeable portion of the moisture exchange tube 10) can receive the proximal end 212 of fitting 200 therein. One way to promote connection of the fitting 200 to the moisture exchange tube 10 is to subject the latter to methanol, which can cause the water-permeable tube 20 to temporarily expand and allow it to engage the proximal end barbs 216. Once the fitting 200 is coupled to the moisture exchange tube 10, the laterally-disposed integral clamps can be brought into cooperation with one another. Referring with particularity to FIG. 4C (in conjunction with FIG. 3) inner surfaces 232, 242 of clamps 230, 240 are rotated about their longitudinal axes to form a latched lock onto the braiding 30 at the end of the moisture exchange tube 10. Thus, the clamps 230, 240 are folded around the body 210 and the end of the moisture exchange tube 10 and snap-fit together, employing detents 244 from one clamp 240 and complementary slots 234 from the other clamp 230 to form a locking connection. By securing the end of the braiding 30, the clamps 230, 240 achieve protection against braiding unravelling, which is particularly valuable as a way to preserve the mechanical integrity of the braiding 30 that in turn provides reinforcement of the moisture exchange tube 10. Significantly, the clamps 230, 240 also provide increased resistance to pull-out of the fitting 200 from the moisture exchange tube 10. By fabricating the fitting 200 separately from the tube 10 and snapping it into place later facilitates ease of assembly, while preserving the high level of mechanical durability.

Referring again to FIG. 5, the fitting 300 only clamps tube 10, while tube 15 may be attached to the opposing end 314 though frictional engagement of its inner surface with one or more barbs (not shown). In another embodiment (not shown) the clamps 330, 340 of fitting 300 can be extended along their axial dimension such that they can clamp around tube 15 placed on end 314; in such an embodiment, the fitting functions as a coupler to both fluidly connect tubes 10 and 15, as well as provide additional attachment security through the action of the clamps. The inner surface of such elongated clamps would be sized and shaped to accommodate the tube 15 and barbs in a manner similar to that shown in FIG. 3 for the barbs 216 at end 212.

Advantages associated with the fitting 200 of the present invention and a fluid-conveying device made in conjunction therewith include simplified fitting and tube manufacture, resulting in a reduced scrap rate. In addition, shorter lengths of Nafion® tubing can be used; since Nafion® and related perfluorinated ionomers are relatively expensive, such reduced use is additionally beneficial. Manufacturing is further simplified by fewer assembly steps and no post-mold trimming. Furthermore, through the use of fitting 200, the need for separate internal tube reinforcement, such as the coupling tube 40 of FIG. 1 is done away with, thereby simplifying the tube construction. Moreover, the ease of attachment to the tubing, hosing or related conduit means that lengths of such conduit can be cut to or otherwise adjusted on-site.

While certain representative embodiments and details have been shown for purposes of illustrating the invention, it will be apparent to those skilled in the art that various changes may be made without departing from the scope of the invention, which is defined in the appended claims.

What is claimed is:

1. A fitting for connecting a fluid handling tube, said fitting comprising:
a generally elongate body defining a fluid bore therethrough such that said bore forms a portion of a flowpath defined in the fluid handling tube, said body comprising:
a fluid inlet defining a proximal end of said fluid bore, said fluid inlet comprising a first connector;
a fluid outlet defining a distal end of said fluid bore and in fluid communication with said fluid inlet, said fluid outlet comprising a second connector; and
a plurality of clamps that are integrally formed with said body through a hinged connection such that upon engagement of said clamps with the fluid handling tube, the fluid handling tube compressively engages with a respective one of said first and second connectors under a force applied by said plurality of clamps such that resistance to leakage and resistance to separation between the fluid handling tube and said fitting are increased.

2. The fitting of claim 1, wherein said plurality of clamps comprise a pair of clamps.

3. The fitting of claim 2, wherein said pair of clamps form a resiliently-biased locking relationship with one another through at least one tab formed on one of said pair of clamps that engages with a complementary slot formed in the other of said pair of clamps.

4. The fitting of claim 2, wherein said pair of clamps extend from said body such that they are substantially radially-opposed to one another.

5. The fitting of claim 2, wherein at least one of said pair of clamps comprises at least one projection disposed on a fluid handling tube-engaging surface thereof, said at least one projection configured to impart a gripping contact with the outer surface of the fluid handling tube.

6. The fitting of claim 5, wherein said at least one projection on each of said clamps cooperate with one another to form a concentric generally circular connection around said body.

7. The fitting of claim 5, wherein said at least one projection on each of said clamps cooperate with one another to form a generally rectangular connection around said body.

8. The fitting of claim 1, wherein at least one of said first and second connectors comprise a barbed end.

9. The fitting of claim 8, wherein said barbed end defines a frustro-conical cross-sectional shape.

10. The fitting of claim 1, wherein said plurality of clamps, upon engagement with one another, are configured to compressively engage the fluid handling tube at one axial end and compressively engage another tube at the other axial end thereof.

11. The fitting of claim 1, wherein said fluid handling tube comprises a fluid drying tube.

12. A fluid-conveying apparatus comprising:
a fluid tube; and
a fitting coupled to said tube, said fitting comprising:
a generally elongate body defining a fluid bore therethrough such that said bore forms a portion of a flowpath defined in said tube, said body comprising a fluid inlet defining a proximal end of said fluid bore and a fluid outlet defining a distal end of said fluid bore and in fluid communication with said fluid inlet; and
a plurality of clamps that are integrally formed with said body through a hinged connection such that upon engagement of said clamps with said fluid tube, said tube compressively engages with a respective one of said proximal and distal ends under a force applied by said plurality of clamps such that resistance to leakage and resistance to separation between said tube and said fitting are increased.

13. The apparatus of claim 12, wherein said tube is configured as a moisture exchange tube comprising a selective and reversible water-absorption inner member, and an outer member disposed about said inner member.

14. The apparatus of claim 13, wherein said outer member is configured to permit direct fluid contact between said inner member and the ambient environment.

15. The apparatus of claim 14, wherein said outer member comprises a braided netting.

16. The apparatus of claim 12, wherein said tube is a component in a medical device.

17. The apparatus of claim 16, wherein said medical device comprises a breathing tube.

18. The apparatus of claim 16, wherein said medical device comprises a medical dryer.

19. The apparatus of claim 12, wherein said tube is a component in gas sample conditioning equipment.

20. A method of attaching a fluid-conveying conduit, said method comprising:
configuring a fitting to comprise a generally elongate body defining a fluid bore therethrough such that said bore forms a flowpath, said body comprising a fluid inlet defining a proximal end of said fluid bore and a fluid outlet defining a distal end of said fluid bore and in fluid communication with said fluid inlet, and a plurality of clamps integrally formed with said body through a hinged connection; and
connecting said fitting to said conduit such that a flowpath formed in said conduit is placed in fluid communication with said fitting flowpath, further such that upon engagement of said plurality of clamps with said conduit, said plurality of clamps cause said conduit to compressively engage with a respective one of said proximal and distal ends of said fitting such that resistance to leakage and resistance to separation of said conduit from said fitting are increased.

21. The method of claim 20, wherein said conduit comprises a tube.

22. The method of claim 21, wherein said tube is a breathing tube.

23. The method of claim 20, wherein said plurality of clamps comprise a pair of clamps that are substantially radially-opposed to one another and comprise at least one projection on an inner surface thereof such that said at least one projection on one of said pair of clamps cooperates with said at least one projection on the other of said pair of clamps to form a concentric profile around said body.

24. The method of claim 20, wherein said plurality of clamps comprise a pair of clamps that are substantially radially-opposed to one another and comprise at least one projection on an inner surface thereof such that said at least one projection on one of said pair of clamps cooperates with said at least one projection on the other of said pair of clamps to form a rectangular profile around said body.

25. The method of claim 20, wherein said plurality of clamps comprise a pair of clamps that are substantially radially-opposed to one another and comprise resiliently-biased latching members that upon engaging form a locking relationship between said pair of clamps.
